(19) United States
(12) Patent Application Publication
(10) Pub. No.: US 2005/0288379 A1
(43) Pub. Date: Dec. 29, 2005

(54) Benzoquinone compounds as anti-cancer agents

(76) Inventors: Xiaoqiang Yan, Shanghai (CN); Yumin Cui, Shanghai (CN); Jianhua Yan, Shanghai (CN); Weihan Zhang, Shanghai (CN); Jifeng Duan, Bedfod, MA (US); Jian Zuo, Shanghai (CN)

Correspondence Address: FISH & RICHARDSON PC, P.O. BOX 1022, MINNEAPOLIS, MN, 55440-1022, US

(21) Appl. No.: 11/156,211

(22) Filed: Jun. 17, 2005

Related U.S. Application Data
(60) Provisional application No. 60/581,684, filed on Jun. 21, 2004.

(51) Int. Cl. A61K 31/12

(52) U.S. Cl. 514/690

(57) ABSTRACT

This invention relates to a method of treating certain cancers by administering an effective amount of a compound of the following formula: 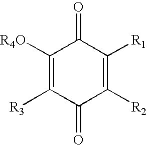
wherein R₁, R₂, R₃, and R₄ are defined herein.

### CROSS REFERENCE TO RELATED APPLICATIONS

Pursuant to 35 USC § 119(e), this application claims priority to U.S. Provisional Application Ser. No. 60/581,684, filed Jun. 21, 2004, the contents of which are incorporated herein by reference.

### BACKGROUND

Cancer, a leading fatal disease, features an abnormal mass of malignant tissue resulting from excessive cell division. Cancer cells proliferate in defiance of normal restraints on cell growth, and invade and colonize territories normally reserved for other cells.

Modes of cancer therapy include chemotherapy, surgery, radiation, and combinations of these treatments. Chemotherapy typically involves use of one or more compounds that inhibit cancer cell growth. While many cancer chemotherapeutic agents have been developed, there remains a need for more effective agents.

### SUMMARY

This invention is based on the discovery that two benzoquinone compounds, i.e., irisqinone A (IqA) and irisquinone B (IqB), effectively inhibit the growth of certain cancer cells.

One aspect of this invention relates to a method for treating cancer, such as esophagus carcinoma, gastric adenocarcinoma, prostate carcinoma, colonic carcinoma, malignant melanoma, or Burkitt's lymphoma. The method includes administering to a subject in need thereof an effective amount of a benzoquinone compound of Formula I: 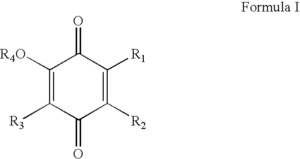
in which R₁ is alkenyl; each of R₂ and R₃, independently, is H, hydroxy, alkoxy, alkyl, or alkenyl; and R₄ is alkyl or aryl.

Referring to Formula I, one subset of the compounds features that R₁ is 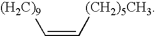
Another subset features that each of R₂ and R₃ is H and R₄ is CH₃.

Another aspect of this invention relates to a method for treating cancer, such as esophagus carcinoma, gastric adenocarcinoma, prostate carcinoma, lung adenocarcinoma, colonic carcinoma, malignant melanoma, Burkitt's lyphoma, breast carcinoma, or hepatoma. The method includes administering to a subject in need thereof an effective amount of a benzoquinone compound of Formula II, 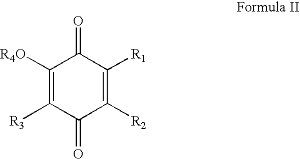
in which R₁ is alkyl; each of R₂ and R₃, independently, is H, hydroxy, alkoxy, alkyl, or alkenyl; and R₄ is alkyl or aryl.

Referring to Formula II, one subset of the compounds features that R₁ is (CH₂)₁₆CH₃. Another subset features that each of R₂ and R₃ is H.

Set forth below are two exemplary compounds that can be used to practice the above methods:

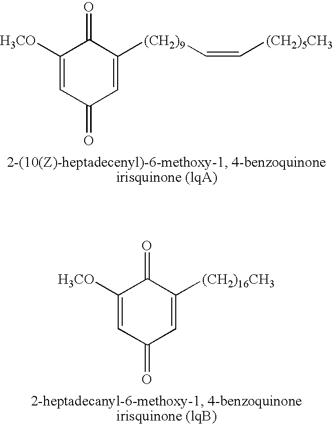

Also within the scope of this invention are (1) a composition containing a compound of Formula I and a pharmaceutically acceptable carrier in treating cancers, such as esophagus carcinoma, gastric adenocarcinoma, prostate carcinoma, colonic carcinoma, malignant melanoma, or Burkitt's lymphoma, as well as the use of such a composition for the manufacture of a medicament for treating the cancers; and (2) a composition containing a compound of Formula II and a pharmaceutically acceptable carrier in treating cancers, such as esophagus carcinoma, gastric adenocarcinoma, prostate carcinoma, lung adenocarcinoma, colonic carcinoma, malignant melanoma, Burkitt's lymphoma, breast carcinoma, or hepatoma, as well as the use of such compositions for the manufacture of medicaments for treating the cancers.

The term “alkyl” refers to a straight or branched hydrocarbon, containing 1-20 carbon atoms. Examples of alkyl groups include, but are not limited to, methyl, ethyl, n-propyl, i-propyl, n-butyl, i-butyl, and t-butyl. The term “alkoxy” refers to an —O-alkyl radical.

The term “alkenyl” refers to a straight or branched hydrocarbon having one or more carbon-carbon double bonds. The alkenyl can contain 1-20 carbon atoms.

The term “aryl” refers to a 6-carbon monocyclic, 10-carbon bicyclic, 14-carbon tricyclic aromatic ring system wherein each ring may have 1 to 4 substituents. Examples of aryl groups include, but are not limited to, phenyl, naphthyl, and anthracenyl.

Alkyl, alkoxy, alkenyl, and aryl mentioned herein include both substituted and unsubstituted moieties. Examples of substituents include, but are not limited to, halo, hydroxyl, amino, cyano, nitro, mercapto, alkoxycarbonyl, amido, carboxy, alkanesulfonyl, alkylcarbonyl, carbamido, carbamyl, carboxyl, thioureido, thiocyanato, sulfonamido, alkyl, alkenyl, alkynyl, alkyloxy, aryl, heteroaryl, cyclyl, and heterocyclyl, in which the alkyl, alkenyl, alkynyl, alkyloxy, aryl, heteroaryl cyclyl, and heterocyclyl may be further substituted.

Details of several embodiments of the invention are set forth in the description below. Other features, objects, and advantages of the invention will be apparent from the description, and also from the claims.

### DETAILED DESCRIPTION

This invention relates to a method of treating esophagus carcinoma, gastric adenocarcinoma, prostate carcinoma, colonic carcinoma, malignant melanoma, or Burkitt's lymphoma using a benzoquinone compound of Formula I, and a method treating esophagus carcinoma, gastric adenocarcinoma, prostate carcinoma, lung adenocarcinoma, colonic carcinoma, malignant melanoma, Burkitt's lyphoma, breast carcinoma, or hepatoma using a benzoquinone compound of Formula II. Each method includes administering to a subject in need thereof an effective amount of a benzoquinone compound. The term “an effective amount” refers to the amount of the active agent that is required to confer the intended therapeutic effect in the subject. Effective amounts may vary, as recognized by those skilled in the art, depending on route of administration, excipient usage, and the possibility of co-usage with other agents. The term “treating” refers to administering one or more of the above-described benzoquinone compounds to a subject that has cancer, or has a symptom of cancer, or has a predisposition toward cancer, with the purpose to cure, heal, alleviate, relieve, alter, remedy, ameliorate, improve, or affect the cancer, the symptoms of the cancer, or the predisposition toward the cancer.

Some of the benzoquinone compounds used to practice this method are naturally occurring and can be isolated from natural sources. For example, IqA and IqB can be isolated from the seed coating of Iris pallasii Fisch. var. chinensis Fisch. and the seed oil of Iris pseudacorus L. Others can be synthesized by methods well known in the art or prepared from the naturally-occurring compounds via simple transformations. The chemicals used in the isolation and synthesis of the benzoquinone compounds may include, for example, solvents, reagents, catalysts, and protecting group and deprotecting group reagents. The isolation and synthesis may also include steps to add or remove suitable protecting groups in order to ultimately obtain desired benzoquinone compounds. Synthetic chemistry transformations and protecting group methodologies (protection and deprotection) useful in synthesizing applicable benzoquinone compounds are known in the art and include, for example, those described in R. Larock, Comprehensive Organic Transformations, VCH Publishers (1989); T. W. Greene and P. G. M. Wuts, Protective Groups in Organic Synthesis, 3^{rd} Ed., John Wiley and Sons (1999); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995) and subsequent editions thereof.

The benzoquinone compounds described above may contain one or more double bonds. Thus, they may occur as cis- or trans-isomeric forms. Such isomeric forms are contemplated.

To practice the methods of this invention, one of the above-described compounds can be applied at the same time or at different times. They can be administered orally, parenterally, by inhalation spray, or via an implanted reservoir. The term “parenteral” as used herein includes subcutaneous, intracutaneous, intravenous, intramuscular, intraarticular, intraarterial, intrasynovial, intrasternal, intrathecal, intralesional and intracranial injection or infusion techniques.

An oral composition can be any orally acceptable dosage form including, but not limited to, tablets, capsules, emulsions and aqueous suspensions, dispersions and solutions. Commonly used carriers for tablets include lactose and corn starch. Lubricating agents, such as magnesium stearate, are also typically added to tablets. For oral administration in a capsule form, useful diluents include lactose and dried corn starch. When aqueous suspensions or emulsions are administered orally, the active ingredient can be suspended or dissolved in an oily phase combined with emulsifying or suspending agents. If desired, certain sweetening, flavoring, or coloring agents can be added.

A sterile injectable composition (e.g., aqueous or oleaginous suspension) can be formulated according to techniques known in the art using suitable dispersing or wetting agents (such as, for example, Tween 80) and suspending agents. The sterile injectable preparation can also be a sterile injectable solution or suspension in a non-toxic parenterally acceptable diluent or solvent, for example, as a solution in 1,3-butanediol. Among the acceptable vehicles and solvents that can be employed are mannitol, water, Ringer's solution and isotonic sodium chloride solution. In addition, sterile, fixed oils are conventionally employed as a solvent or suspending medium (e.g., synthetic mono- or di-glycerides). Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions can also contain a long-chain alcohol diluent or dispersant, or carboxymethyl cellulose or similar dispersing agents.

An inhalation composition can be prepared according to techniques well known in the art of pharmaceutical formulation and can be prepared as solutions in saline, employing benzyl alcohol or other suitable preservatives, absorption promoters to enhance bioavailability, fluorocarbons, and/or other solubilizing or dispersing agents known in the art.

A topical composition can be formulated in form of oil, cream, lotion, ointment and the like. Suitable carriers for the composition include vegetable or mineral oils, white petrolatum (white soft paraffin), branched chain fats or oils, animal fats and high molecular weight alcohols (greater than C12). The preferred carriers are those in which the active ingredient is soluble. Emulsifiers, stabilizers, humectants and antioxidants may also be included as well as agents imparting color or fragrance, if desired. Additionally, transdermal penetration enhancers may be employed in these topical formulations. Examples of such enhancers can be found in U.S. Pat. Nos. 3,989,816 and 4,444,762. Creams are preferably formulated from a mixture of mineral oil, self-emulsifying beeswax and water in which mixture the active ingredient, dissolved in a small amount of an oil, such as almond oil, is admixed. An example of such a cream is one which includes about 40 parts water, about 20 parts beeswax, about 40 parts mineral oil and about 1 part almond oil. Ointments may be formulated by mixing a solution of the active ingredient in a vegetable oil, such as almond oil, with warm soft paraffin and allowing the mixture to cool. An example of such an ointment is one which includes about 30% almond and about 70% white soft paraffin by weight.

A carrier in a pharmaceutical composition must be “acceptable” in the sense of being compatible with the active ingredient of the formulation (and preferably, capable of stabilizing it) and not deleterious to the subject to be treated. For example, solubilizing agents, such as cyclodextrins (which form specific, more soluble complexes with one or more of active compounds of the extract), can be utilized as pharmaceutical excipients for delivery of the active compounds. Examples of other carriers include colloidal silicon dioxide, magnesium stearate, cellulose, sodium lauryl sulfate, and D&C Yellow # 10.

Suitable in vitro assays can be used to preliminarily evaluate the efficacy of the above-described compound in inhibiting proliferation of cancer cells. More specifically, a test compound can be added to cancer cells and its IC₅₀ value (i.e., the concentration of the test compound required to reach a half-maximal inhibition of cell growth) is determined. The active compound can further be examined for its efficacy in treating cancer by in vivo assays. For example, the compound can be administered to an animal (e.g., a mouse model) having cancer and its therapeutic effects are then accessed. Based on the results, an appropriate dosage range and administration route can also be determined.

Without further elaboration, it is believed that the above description has adequately enabled the present invention. The following specific example is, therefore, to be construed as merely illustrative, and not limitative of the remainder of the disclosure in any way whatsoever. All of the publications cited herein are hereby incorporated by reference in their entirety.

Inhibition of Cancer Cell Growth

An in vitro assay was conducted to evaluate the efficacy of IqA and IqB in inhibiting the growth of cancer cells.

Human cancer cell lines, i.e., Eca-109 (esophagus carcinoma cell line), AGS and BGC-823 (gastric adenocarcinoma cell lines), DU145 (prostate carcinoma cell line), SPC-A-1 (lung adenocarcinoma cell line), Caco-2 and LS-174T (colonic carcinoma cell lines), A375 (malignant melanoma cell line), Raji (Burkitt's lymphoma cell line), MCF-7 and Bcap-37 (breast carcinoma cell lines), HepG2 (hepatoma cell line), were purchased from the Cell Bank of Shanghai Institute of Cell Biology, Chinese Academy of Sciences, and cultured in Iscove's Modified Dulbecco's Medium (IMDM) containing 10% fetal bovine serum (FBS) in an incubator at 37° C. under 5% CO₂. Cells of 70-80% confluence were trypsinized, resuspended in IMDM medium containing 10% FBS at 1×10⁵ cells/ml, and seeded in 96-well plates (100 μl in each well). The plates were incubated at 37° C. under 5% CO₂ overnight.

IqA and IqB were provided by Shandong Xinhua Pharmaceutical CO. Ltd. IqA and IqB stock solutions were prepared at the concentrations of 10 mg/ml and 1 mg/ml in dimethyl sulfoxide (DMSO), respectively. The IqA solution was diluted with the growth medium into a series of solutions at different concentrations. The diluted solutions (10 μl) were added to wells containing cancer cells. The final concentrations in the wells were 0.3, 1, 3, 10, 30, and 100 μg/ml. Similarly, the IqB solution was diluted and added to wells to reach the final concentrations of 0.03, 0.1, 0.3, 1, 3, and 10 μg/ml. 10 μl of DMSO was added to wells containing cancer cells and these wells were used as the control. Wells to which none of IqA, IqB, and DMSO was added served as the background. The plates were then incubated at 37° C. under 5% CO₂ for 48 hrs.

10 μl of 5 mg/ml 3-(4,5-dimethylthiazol-2-yl)-2,5-diphenyltetrazolium bromide was each added to all wells except for the background wells. After additional incubation for 3-4 hrs, the plates were spun at 1000 rpm for 15 minutes and the supernatants were carefully removed by vacuum. The cells were washed with 150 μl of phosphate-buffered saline.

150 μl of DMSO was added to each well. The plates were placed on a shaker at 150 rpm for 15 minutes to dissolve the precipitate in the wells. Absorbance was measured at 492 nm using a microplate reader. Experiments were done in triplicate.

A software program, XLfit (ID Business Solutions), was used to calculate the IC₅₀ values of IqA and IqB against the growth of the above-mentioned cancer cells. The results show that both IqA and IqB effectively inhibited the proliferation of these cancer cells.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

1. A method for treating cancer, comprising administering to a subject in need thereof an effective amount of a compound of Formula I: 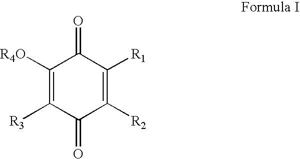
in which R₁ is alkenyl; each of R₂ and R₃, independently, is H, hydroxy, alkoxy, alkyl, or alkenyl; and R₄ is alkyl, or aryl;
wherein the cancer is esophagus carcinoma, gastric adenocarcinoma, prostate carcinoma, colonic carcinoma, malignant melanoma, or Burkitt's lymphoma.

2. The method of claim 1, wherein the cancer is esophagus carcinoma.

3. The method of claim 1, wherein the cancer is gastric adenocarcinoma.

4. The method of claim 1, wherein the cancer is prostate carcinoma.

5. The method of claim 1, wherein the cancer is colonic carcinoma.

6. The method of claim 1, wherein the cancer is malignant melanoma.

7. The method of claim 1, wherein the cancer is Burkitt's lymphoma.

8. The method of claim 1, wherein R₁ is

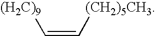

9. The method of claim 8, wherein each of R₂ and R₃ is H.

10. The method of claim 9, wherein R₄ is CH₃.

11. The method of claim 1, wherein each of R₂ and R₃ is H.

12. A method for treating cancer, comprising administering to a subject in need thereof an effective amount of a compound of Formula II: 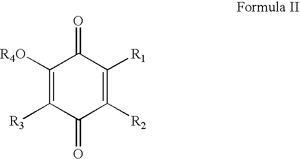
in which R₁ is alkyl; each of R₂ and R₃, independently, is H, hydroxy, alkoxy, alkyl, or alkenyl; and R₄ is alkyl, or aryl;
wherein the cancer is esophagus carcinoma, gastric adenocarcinoma, prostate carcinoma, lung adenocarcinoma, colonic carcinoma, malignant melanoma, Burkitt's lymphoma, breast carcinoma, or hepatoma.

13. The method of claim 12, wherein the cancer is esophagus carcinoma.

14. The method of claim 12, wherein the cancer is gastric adenocarcinoma.

15. The method of claim 12, wherein the cancer is prostate carcinoma.

16. The method of claim 12, wherein the cancer is lung adenocarcinoma.

17. The method of claim 12, wherein the cancer is colonic carcinoma.

18. The method of claim 12, wherein the cancer is malignant melanoma.

19. The method of claim 12, wherein the cancer is Burkitt's lymphoma.

20. The method of claim 12, wherein the cancer is breast carcinoma.

21. The method of claim 12, wherein the cancer is hepatoma.

22. The method of claim 12, wherein R₁ is (CH₂)₁₆CH₃.

23. The method of claim 22, wherein each of R₂ and R₃ is H.

24. The method of claim 23, wherein R₄ is CH₃.

25. The method of claim 12, wherein each of R₂ and R₃ is H.
